(19) United States
(12) Patent Application Publication
(10) Pub. No.: US 2008/0139903 A1
(43) Pub. Date: Jun. 12, 2008

(54) METHOD AND APPARATUS FOR INSERTION OF A SENSOR USING AN INTRODUCER

(75) Inventors: Robert Bruce, Beaverton, OR (US); W. Kenneth Ward, Portland, OR (US); Richard G. Sass, Portland, OR (US); Jon Fortuna, Mechanicsburg, PA (US); Mark Neinast, Lake Oswego, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: ISENSE CORPORATION, Wilsonville, OR (US)

(21) Appl. No.: 11/952,033

(22) Filed: Dec. 6, 2007

Related U.S. Application Data
(60) Provisional application No. 60/869,288, filed on Dec. 8, 2006.

(51) Int. Cl. A61B 17/34

(52) U.S. Cl. 600/309; 600/373; 606/129

(57) ABSTRACT

A device and method for delivering a device such as a sensor or fluid transport structure or a fluid transport structure sensor combination into, for example, animal skin. Such a device utilizes an introducer device such as a needle or trocar to puncture skin and allow a sensor to enter through the resultant skin puncture. A device in accordance with embodiments of the present invention includes a housing for mounting to animal skin including an opening for receiving both the distal end of an analyte sensor and the end of an introducer, an actuator device for forcing the introducer from a first position within the housing, through the exit port to a second position, with sufficient force to puncture skin.

### CROSS REFERENCE TO RELATED APPLICATIONS

The present application claims priority to U.S. Provisional Patent Application No. 60/869,288, filed Dec. 8, 2006, entitled “Method and Apparatus for Insertion of a Sensor Using an Introducer,” the entire disclosure of which is hereby incorporated by reference in its entirety.

### TECHNICAL FIELD

This present invention relates generally to devices for delivering mechanically slender devices through skin into a body to perform various medical or physiological functions. More specifically the present invention relates to a method for transcutaneous placement of a soft cannula biosensor or flexible biosensor safely and automatically, and aided by a rigid and/or sharp introducer device deployed using manual or automatic means.

### BACKGROUND

There are several instances of medically useful devices which are mechanically slender and flexible and are also inserted through the skin. For example, sensors facilitate the sensing of certain conditions within a patient. Electrochemical sensors are commonly used to monitor blood glucose levels in the management of diabetes. In one scheme, an electrochemical sensor incorporating an enzyme is fabricated onto a small diameter wire. A second reference electrode is also fabricated around the wire near the sensing electrode. The sensor assembly is inserted through the skin so that it is surrounded by interstitial fluid. A portion of the sensor assembly exits the skin, remaining outside the body, where electrical connections to the sensing electrode and reference electrode are present or may be made. A suitable electronic measuring device outside the body may be used to measure electrical current from the sensor for recording and display of a glucose value. These types of devices are described, for example, in U.S. Pat. No. 5,965,380 to Heller et al. and U.S. Pat. No. 5,165,407 to Wilson et al.

In addition to electrochemical glucose sensors, a number of other electrochemical sensors have been developed to measure the chemistry of blood or other body fluids or materials. Electrochemical sensors generally make use of one or more electrochemical processes and electrical signals to measure a parameter. Other types of sensors include those which use optical techniques to perform a measurement.

In other applications, a cannula and sensor combination device is inserted through the skin to allow insulin to be introduced into the body as part of an artificial pancreas system. In these applications, a slender (small cross-section) and flexible device offers several advantages over a larger and more rigid device. Patient comfort is increased, especially during long-term insertion, and trauma at the entry site is reduced. A flexible device also is able to adjust to movement of the skin during physical activity, increasing patient comfort. In many cases these devices will remain inserted in the body for 5 to 7 days.

Although the slender and flexible nature of these devices increases patient comfort, these devices are difficult to insert through the skin. Unlike a typical hypodermic needle, these devices are generally too fragile and flexible to be simply pushed through the skin surface using normal force and speed. When the tip of such a device is forced against the skin, the device may bend and collapse with much less force than may be required to achieve skin penetration. Although in some cases the tip of the device may be sharpened to ease penetration, this approach is not typically adequate to assure penetration, and some devices such as tubing-based devices are not appropriate for sharpening. Also, the sharpening process adds to production cost and complexity.

As will be understood by those skilled in the art, human skin possesses biomechanical properties influenced by a relatively impenetrable outer layer, the stratum corneum, and inner layers which are more easily penetrated. These biomechanical properties cause penetration of the skin surface to present the primary challenge in introducing a relatively fragile slender, flexible device into the skin.

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIG. 1 illustrates a block diagram of an insertion device according to embodiments of the present invention;

FIG. 2A illustrates an embodiment of an electrochemical glucose sensor that has been fabricated onto a length of thin, flexible wire in accordance with embodiments of the present invention;

FIG. 2B shows a cross-section of how an electrochemical sensor may appear when inserted into skin in accordance with an embodiment of the present invention;

FIG. 3 shows a rotating cam actuator device for sensor insertion using an introducer according to embodiments of the invention;

FIG. 4A shows, according to embodiments of the invention, components of a rotating cam actuator in its pre-insertion configuration and laterally separated to illustrate motion;

FIG. 4B shows, according to embodiments of the invention, components of a rotating cam actuator during the introducer insertion phase and laterally separated to illustrate motion;

FIG. 4C shows, according to embodiments of the invention, components of a rotating cam actuator after introducer insertion and laterally separated to illustrate motion;

FIG. 4D shows, according to embodiments of the invention, components of a rotating cam actuator during sensor insertion and laterally separated to illustrate motion;

FIG. 4E shows, according to embodiments of the invention, components of a rotating cam actuator after sensor insertion and laterally separated to illustrate motion;

FIG. 5A shows an introducer device for insertion of an introducer either simultaneously with or shortly before insertion of a sensor in accordance with embodiments of the invention;

FIG. 5B shows a cross-sectional view of an introducer device for insertion of an introducer either simultaneously with or prior to insertion of a sensor in accordance with embodiments of the invention;

FIG. 5C shows an introducer device with the body of the device removed exposing the interior components of the device in accordance with embodiments of the invention;

FIG. 6 shows a rotating cam actuator mated to an introducer device in accordance with embodiments of the invention;

FIG. 7A shows a push-button introducer device utilizing a “U” shaped feature of a spring member to puncture skin prior to sensor insertion in accordance with embodiments of the invention;

FIG. 7B shows a cross-sectional view of a push-button introducer device in accordance with embodiments of the invention;

FIG. 8A shows a cross-sectional view of a cantilevered introducer device utilizing a cantilever spring for introducer insertion either simultaneous with or prior to sensor insertion in accordance with embodiments of the invention;

FIG. 8B shows a cantilevered introducer device with the device body removed to expose its interior components in accordance with embodiments of the invention;

FIG. 9 shows a manual introducer pre-set actuator utilizing a two-button manual actuator design for introducer and sensor insertion according to embodiments of the invention; and

FIG. 10 shows a cross-sectional view of a cantilevered introducer device mated to a manual introducer pre-set actuator device in accordance with embodiments of the invention.

### DETAILED DESCRIPTION OF THE EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description (including the claims) may use perspective-based descriptions such as up/down, back/front, and top/bottom. Such descriptions are merely used to facilitate the discussion and are not intended to restrict the application of embodiments of the present invention.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

The term “actuator” refers to any of various electric, hydraulic, magnetic, pneumatic, or other means by which something is moved or controlled.

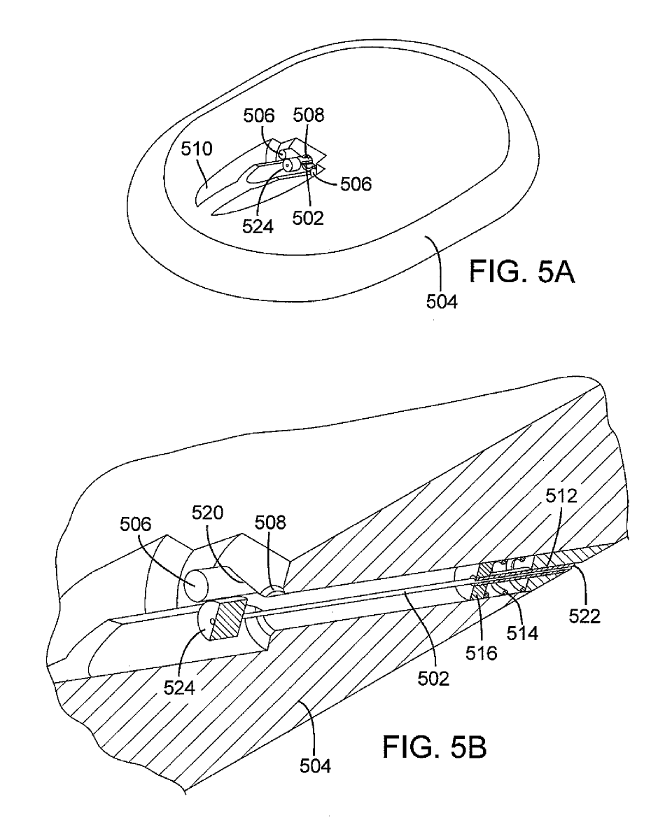

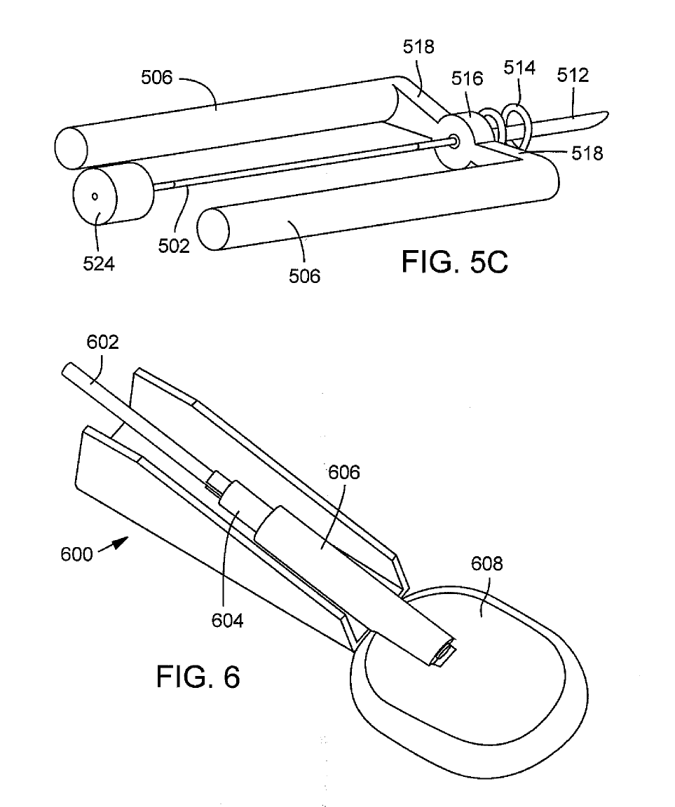

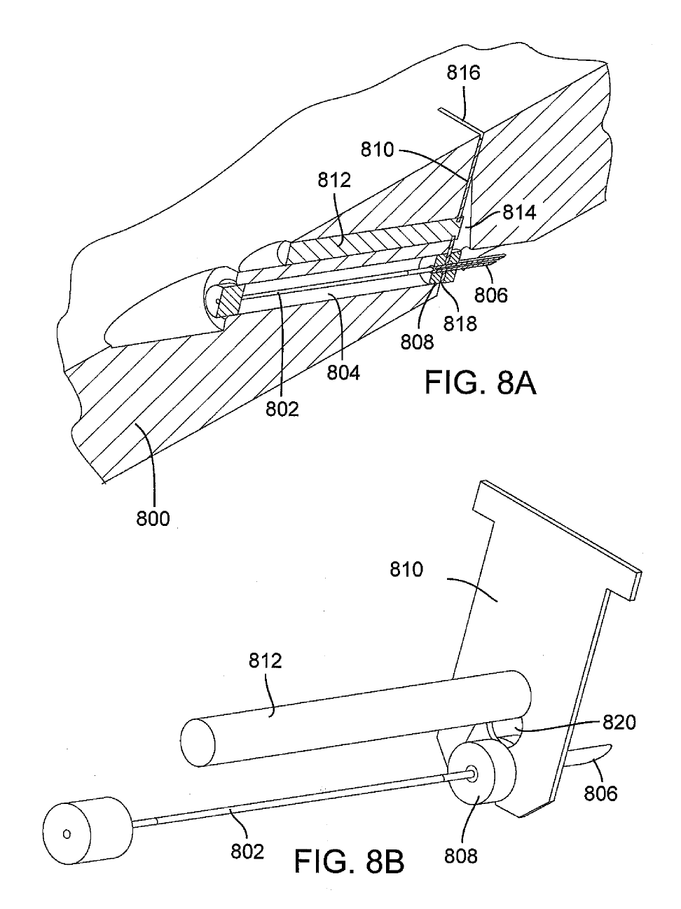

The phrase “sensor insertion guidance structure” means a physical structure that either guides an analyte sensor in a pre-determined direction, provides axial support to the sensor upon application of motive force to the sensor, or both. Such axial support may be provided either directly to an analyte sensor or indirectly to the sensor by providing direct support to a guide member attached to the sensor. Examples include, but are not limited to, cylindrical channel 508 shown in FIGS. 5A-5C, channel 708 shown in FIG. 7B, and channel 804 shown in FIG. 8A. Sensor insertion guidance structures need not be cylindrical. Additional details about suitable guidance structures may be found in U.S. patent application Ser. No. 11/558,394, filed Nov. 9, 2006, the entire contents of which are hereby incorporated by reference. Although various embodiments of the present invention involve insertion of a sensor using an introducer, details of guidance structure designs from U.S. patent application Ser. No. 11/558,394 may be extended to various embodiments of the present invention.

The term “axial support” means the support or bracing of a relatively straight, slender object when a motive force is applied to the object in such a way as to resist force vectors acting perpendicular to an imaginary line drawn through the device lengthwise; such support or bracing sufficient to prevent or reduce crimping, creasing, folding, or bending of the straight, slender object; or such support or bracing sufficient to enable the object to return to a relatively straight configuration after minimal bending such that the object substantially retains its original shape with minimal crimping, creasing, folding, or bending.

The term “guide member” means a device that at least partially axially surrounds the analyte sensor and is adapted to fit inside the guidance structure such that the guide member at least partially occupies at least some part of the space between the sensor and the guidance structure during insertion, before insertion, and/or after insertion. A guide member may provide axial support or assist a sensor in moving through the guidance structure, or both. Examples include cylindrical sensor guide 524 shown in FIGS. 5A-5C. Other exemplary guide members include a spiral of plastic, a rectangular metallic guide, an open cell foam plastic cylinder, and a thin plastic disk. As will be appreciated by one of ordinary skill in the art, a guide member may be made of many different materials and shaped in various geometries which may or may not correspond to the geometry of the guidance structure. Additional details about suitable guide members may be found in U.S. patent application Ser. No. 11/558,394, filed Nov. 9, 2006, the entire contents of which are hereby incorporated by reference. Although various embodiments of the present invention involve insertion of a sensor using an introducer, details of guide member designs from U.S. patent application Ser. No. 11/558,394 may be extended to various embodiments of the present invention.

For the purposes of describing embodiments of the present invention and in the claims that follow, the term “electrical network” means electronic circuitry and components in any desired structural relationship adapted to, in part, receive an electrical signal from an associated sensor and, optionally, to transmit a further signal, for example to an external electronic monitoring unit that is responsive to the sensor signal. The circuitry and other components may or may not include a printed circuit board, a tethered or wired system, etc. Signal transmission may occur over the air with electromagnetic waves, such as RF communication, or data may be read using inductive coupling. In other embodiments, transmission may be over a wire or via another direct connection.

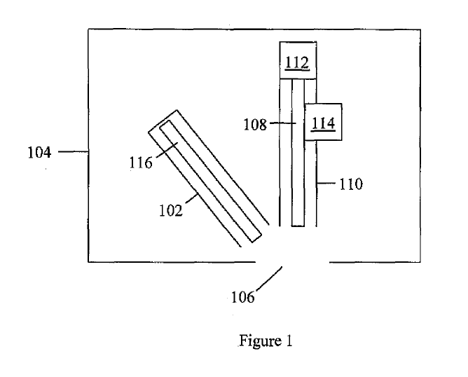

An embodiment of the present invention may include, as shown in FIG. 1, sensor insertion guidance structure 102, coupled to housing 104 and oriented such that sensor insertion guidance structure 102 may allow the passage of analyte sensor 116 through opening 106. Introducer 108 may be situated inside support structure 110 which may provide support and guidance to introducer 108. Actuator 112 may be placed in proximity to introducer 108 and may be adapted to provide a motive force to introducer 108 such that it moves through support structure 110 and thus may be inserted into animal skin (not shown) causing a skin puncture. In an embodiment, a motive force device (not shown) separate from or the same as actuator 112 may be provided to drive sensor 116 into skin when in use. Retraction mechanism 114 may also be coupled to introducer 108 and may provide a means, for example an automatic means, by which introducer 108 is retracted from the skin and pulled back inside housing 104 of the device after insertion of introducer 108. In an embodiment, retraction mechanism 114 may be a separate device from actuator 112. In an alternative embodiment, retraction mechanism 114 may be the same or part of the same device as actuator 112 (for example a spring). In an embodiment, a retraction mechanism separate from or the same as retraction mechanism 114 may be provided to retract sensor 116 from skin.

In embodiments, the introducer may be hollow. In embodiments, the analyte sensor may be situated such that it may be inserted into skin by passing through a hollow introducer. In embodiments, the introducer may be inserted prior to sensor insertion and retracted before the sensor may enter the skin through the puncture created by the introducer. In embodiments, the introducer may be inserted prior to sensor insertion and retracted after the sensor has been separately inserted through the introducer into the skin. In embodiments, the introducer may be inserted simultaneously with sensor insertion. In embodiments, the introducer may remain in the skin of the patient after insertion of the sensor. In embodiments, the housing including the introducer may be withdrawn from the skin after sensor insertion leaving only the exposed sensor partially inserted into the skin and appropriate electrical connections may be made directly to the sensor, or, in an embodiment, such connections may already be present. In embodiments, the housing may be removed leaving both the introducer and the sensor partially inserted into the skin.

In embodiments, the introducer may be inserted into the skin to a depth of between 1 mm and 5 mm, for example approximately 2 mm. In alternative embodiments, the introducer may be inserted at depths less than 1 mm and/or greater than 5 mm.

In embodiments, an introducer actuation device may be any of various electric, hydraulic, magnetic, pneumatic, or manual actuator devices including, for example, linear solenoid actuators, rotary solenoid actuators, pressurized gas (such as CO₂) cartridge actuators, spring actuators, air pump actuators, etc. Additional details about suitable actuators may be found in U.S. patent application Ser. No. 11/558,394, filed Nov. 9, 2006, the entire contents of which are hereby incorporated by reference, and which describes a method and apparatus for insertion of an analyte sensor without an introducer. Although embodiments of the present invention involve insertion of a sensor using an introducer, details of an actuator design from U.S. patent application Ser. No. 11/558,394 may be extended to embodiments of the present invention.

In embodiments, a sensor may be inserted via a sensor actuator device (not shown in FIG. 1) including any of various electric, hydraulic, magnetic, pneumatic, or manual actuator devices including, for example, linear solenoid actuators, rotary solenoid actuators, pressurized gas (such as CO₂) cartridge actuators, spring actuators, air pump actuators, etc. Additional details about suitable actuators may be found in U.S. patent application Ser. No. 11/558,394, which describes a method and apparatus for insertion of an analyte sensor without an introducer. Although embodiments of the present invention involve insertion of a sensor using an introducer, details of an actuator design from patent application Ser. No. 11/558,394 may be extended to embodiments of the present invention.

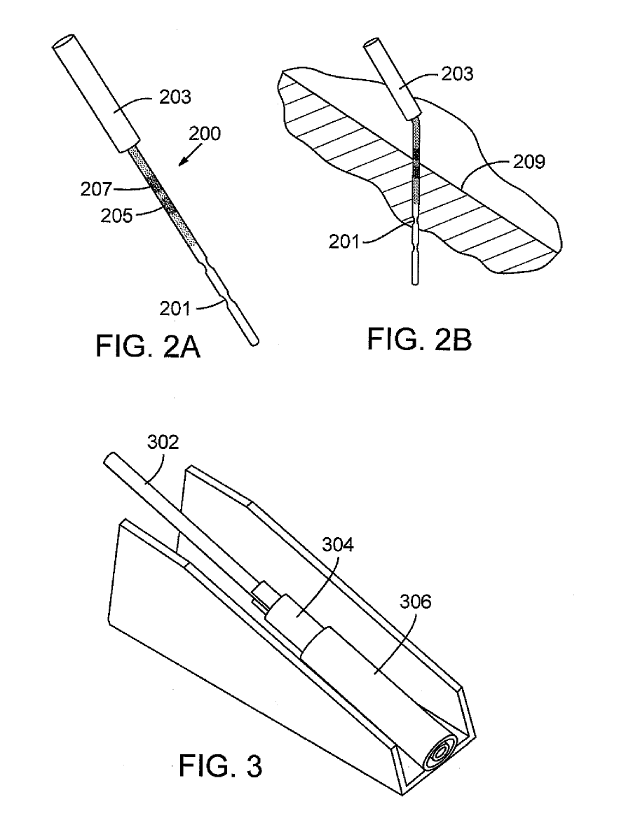

FIG. 2A shows an analyte sensor 200 that may be inserted according to various embodiments of the present invention. In FIG. 2A, analyte sensor 200 is an electrochemical glucose sensor that has been fabricated onto a length of thin, flexible wire. A reference or ground electrode 205 and a sensing electrode 207 may be incorporated into analyte sensor 200. Small diameter end 201 (proximal end) of sensor 200 may be inserted through the skin. In an embodiment, small diameter end 201 may have a diameter of approximately 0.25 mm or less. In an embodiment, on the larger diameter end (distal end) of sensor 200, its diameter has been increased by adding a sleeve of tubing 203, such as steel tubing, which may increase its rigidity and facilitate electrical connections. In some embodiments, the diameter of the larger section may be, for example, approximately 0.5 mm. In an embodiment, the larger diameter portion of the sensor may remain outside of the body upon insertion. In an embodiment, sensor 200 may be formed, for example, by etching away material such as dielectric, etc. FIG. 2B shows a cross-section of the sensor when inserted into skin 209. In some embodiments, a 10-20 mm, for example approximately 15 mm, length of sensor 200 may be implanted beneath skin 209.

In embodiments, a sensor inserted according to an embodiment of the present invention may be rigid or flexible. In some embodiments, a flexible sensor is one that may be flexed repeatedly, such as the type of flexion experienced by a subcutaneously implanted sensor in a human during normal movement, over a period of time (such as 3-7 days or more) without fracture. In an embodiment, a flexible sensor may be flexed hundreds or thousands of times without fracture.

FIG. 3 shows a rotating cam actuator in accordance with various embodiments of the present invention. Center rod 302 and sleeve 304 may be inserted inside hollow tube 306. Center rod 302 may be adapted to transmit motive force for insertion of a sensor (not shown) while sleeve 304 may be adapted to transmit motive force for insertion of an introducer (not shown).

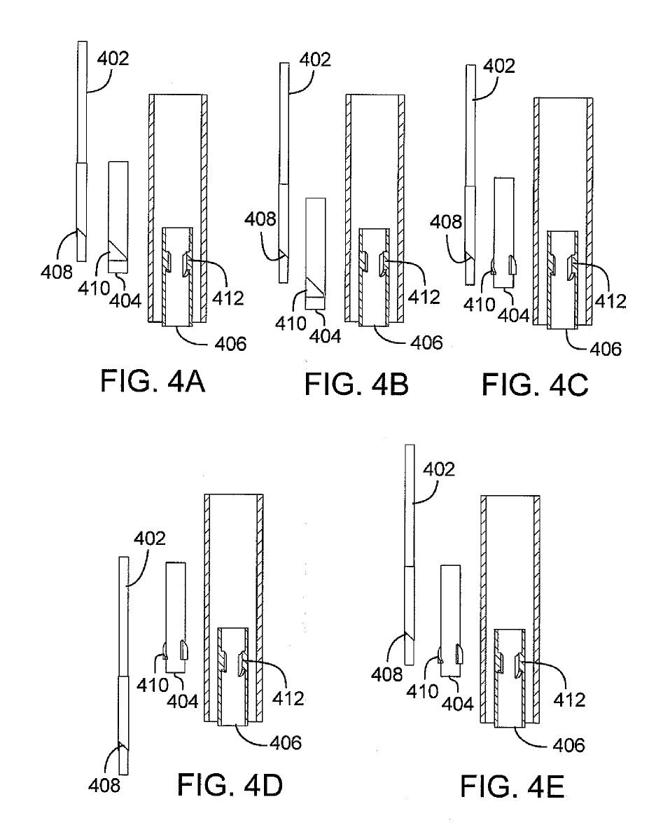

FIGS. 4A-4F show components of a rotating cam actuator in accordance with various embodiments of the present invention laterally separated to illustrate how the components may cooperate during introducer and sensor insertion. FIG. 4A shows the components just before introducer insertion. Center rod 402 may have exterior cam surfaces 408, while sleeve 404 may have both interior cam surfaces (not shown) and exterior cam surfaces 410. Hollow tube 406 may have interior cam surfaces 412. Center rod exterior cam surfaces 408 may be adapted to interfere with interior sleeve cam surfaces (not shown) and the surfaces may, in an embodiment, be in contact on an angle. Also, exterior sleeve cam surfaces 410 may be adapted to interfere with hollow tube interior cam surfaces 412.

Shown in FIG. 4B, initially applying force to the top of center rod 402 in a downward direction may drive center rod 402 as well as sleeve 404 downward due to the interference between center rod exterior cam surface 408 and the sleeve interior cam surface (not shown).

Shown in FIG. 4C, once sleeve 404 has traveled a certain distance, its exterior cam surface 410 may slip past hollow tube inside cam surface 412 allowing sleeve 404 to rotate. Upon rotation, the sleeve inner cam surface may move off of center rod exterior cam surface 408 and be freed from the continued travel of center rod 402.

Shown in FIG. 4D, upon being freed from center rod 402, sleeve 404 may remain stationary while center rod 402 continues its downward travel. If center rod 402 is adapted for sensor insertion and sleeve 404 is adapted for introducer insertion, the initial movement of sleeve 404 may force an introducer (not shown) into skin for the length of travel of sleeve 404. Meanwhile, the continued travel of center rod 402 may cause a sensor (not shown) to be inserted into the skin to a depth deeper than the depth that the introducer may be inserted. In embodiments, the introducer may be hollow and the sensor may be adapted to pass through the interior of the introducer to enter the skin. In other embodiments, a hollow introducer may be adapted to be retracted prior to sensor insertion and the sensor may be adapted to enter the skin through the interior of the hollow introducer and enter the skin through the skin puncture caused by the introducer. In other embodiments, the sensor may enter the skin through the skin puncture without passing through a hollow introducer. In those embodiments, the introducer may or may not be hollow.

Center rod 402 is shown retracted after sensor insertion in FIG. 4E. Retraction may be accomplished automatically by a retraction spring (not shown), by user intervention, or other means. When center rod 402 has retracted to its original position, center rod exterior cam surface 408 and inner sleeve cam surface (not shown) may no longer be interfering and the sleeve may be free to rotate back to its initial location as shown in FIG. 4A. At this point, in various embodiments, the actuator may be ready for its next insertion.

FIGS. 5A-5C show an introducer device for insertion of an introducer either before or simultaneously with the insertion of a sensor in accordance with various embodiments of the present invention. FIG. 5A shows the top surface of such a device. Sensor 502 is shown with cylindrical sensor guide 524 at its top end. Sensor guide 524 may be adapted to help guide sensor 502 through sensor insertion channel 508 upon sensor insertion. Pins 506 may be adapted to actuate an introducer (not shown) contained within housing 504. Slot 510 may be adapted to allow attachment of an actuator device which may provide motive force for insertion of sensor 502 and/or an introducer (not shown).

In embodiments, housing 504 may additionally contain circuitry which may include an electrical network adapted to receive an electrical signal from sensor 502 and to transmit a further signal, for example to an external electronic monitoring unit or other electrically coupled system that is responsive to the sensor signal. In embodiments, an electrical network may comprise a variety of components in any desired structural relationship, whether or not the network has a printed circuit board, a tethered or wired system, etc. In an embodiment, signal transmission may occur over the air with electromagnetic waves, such as RF communication, or data may be read using inductive coupling. In other embodiments, transmission may be over a wire or via another direct connection.

FIG. 5B shows a cross section of a device for insertion of an introducer and/or a sensor in accordance with various embodiments of the present invention. Housing 504 is shown in cross-section exposing a cross-sectional view of sensor insertion channel 508 with sensor 502 contained therein. The lower portion of sensor 502 may be situated inside hollow introducer 512 near the end of sensor insertion channel 508 and positioned near retraction spring 514. In an embodiment, introducer 512 may be molded into introducer insert molding 516.

Referring now to FIG. 5C, introducer insert molding 516 may be connected to pins 506 via brackets 518. Brackets 518 may travel in two narrow slots 520 located along the sides of sensor insertion channel 508 (seen in FIG. 5B). Prior to actuation, the device may be placed in proximity to (close to or against) animal skin. Referring back to FIG. 5B, the heads of pins 506 may be pushed into the device by an actuation device (not shown). This movement of pins 506 may force introducer 512 to exit via opening 522, puncture the animal skin located below opening 522, and then be inserted into the skin. Upon introducer insertion, retraction spring 514 may exert force on introducer insert molding 516 which may force introducer 512 to retract and exit the skin. In embodiments, sensor 502 may be subsequently driven through the hollow portion of retracted introducer 512 and inserted into the skin via the skin puncture caused by introducer 512. In alternative embodiments, sensor 502 may be inserted into skin while introducer 512 remains inserted. In an embodiment, sensor 502 may be inserted at the same time as introducer 512, and the subsequent retraction of introducer 512 may allow sensor 502 to remain inserted in the skin.

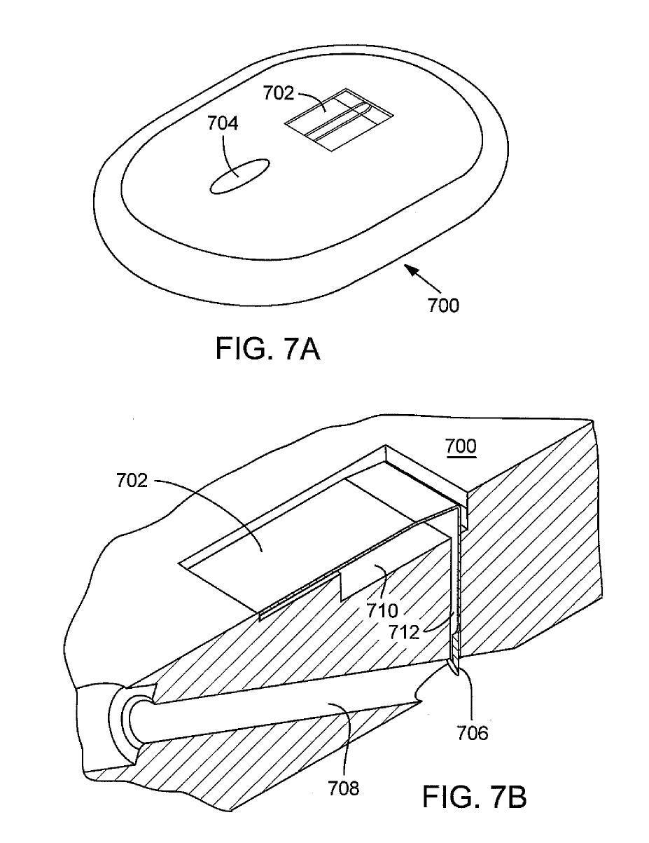

FIG. 6 shows the mating of a rotating cam actuator and an introducer device for introducer insertion in accordance with an embodiment of the present invention. Rotating cam actuator 600 is shown with hollow tube 606, sleeve 604, and center rod 602. The lower end of hollow tube 606 may be mated to introducer insertion device 608 via slots (not shown). To insert an introducer and sensor, center rod 602 may be pressed downward, causing sleeve 604 to move downward for a short distance before it becomes stationary and allows center rod 602 to continue its downward path (see, for example, descriptions of FIGS. 3 and 4A-4F above). Upon insertion, sleeve 604 may come into contact with two pins (not shown) which may be mated to introducer insert molding (not shown—see description of FIGS. 5A-5C above). Thus, while sleeve 604 is moving downward, an introducer may be forced out of the bottom of the device and inserted into skin to a certain depth. Also, after sleeve 604 becomes stationary but center rod 602 continues downward, center rod 602 may contact the end of a sensor which may force the sensor to pass through the hollow portion of an introducer and be inserted into the skin through the skin puncture caused by the introducer and, in an embodiment, to a greater depth than the inserted introducer. In embodiments, the introducer may be retracted while the sensor remains in place, or may be retracted prior to sensor insertion.

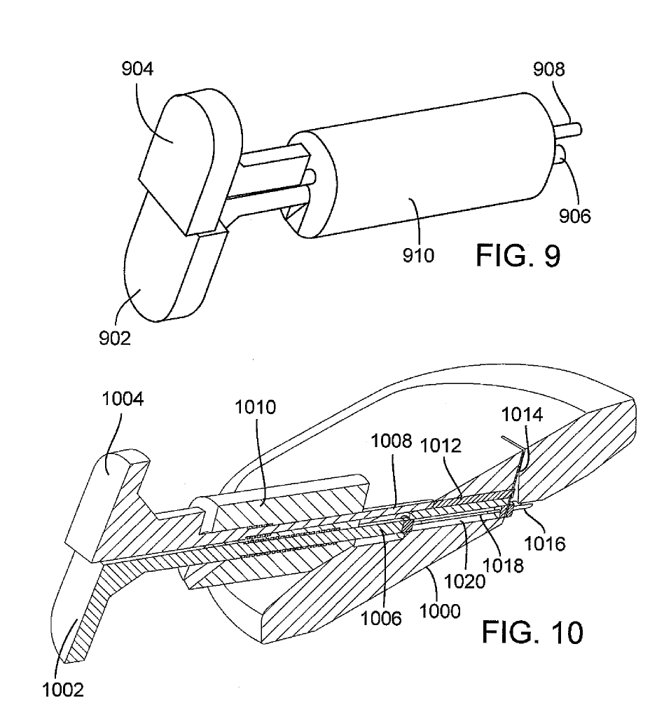

Although a rotating cam actuator is shown mated to an introducer device in FIG. 6, it will be appreciated by one of ordinary skill in the art that in other embodiments, alternative actuator devices may be mated to an introducer device. Alternatives include, for example, a manual pre-set actuator described below and depicted in FIG. 9 or a similar device. Additionally, an introducer may be inserted using any of various electric, hydraulic, magnetic, pneumatic, or manual actuator devices including, for example, linear solenoid actuators, rotary solenoid actuators, CO₂ cartridge actuators, spring actuators, and air pump actuators, etc.

FIGS. 7A and 7B show a push button introducer device in accordance with an embodiment of the present invention. Referring to FIG. 7A, the top surface of spring 702 may be located on the top of device 700 along with port 704 adapted for insertion of a sensor (not shown) into device 700.

Referring to FIG. 7B, spring 702 may be a stamped stainless steel member with a second surface formed at an angle with respect to a first surface. The second surface may be situated inside slot 712. In an embodiment, at the tip of the second surface may be introducer 706, such as a pointed, sharp, U-shaped, or V-shaped feature, adapted to puncture skin. Sensor insertion channel 708 may be adapted to guide a sensor (not shown) into skin. If force is applied to the top surface of spring 702 introducer 706 may be driven downward out of the bottom of device 700 and puncture the skin. Space 710 allows appropriate flexion/depression of spring 702. Whenever force is removed from spring 702, it may return to its initial position which may retract introducer 706 and pull it back out of the skin.

In an embodiment, a sensor may subsequently pass through sensor insertion channel 708 and into the skin through the resultant skin puncture. In another embodiment, the insertion channel may be angled such that the shape at the tip of the introducer may allow the sensor to be inserted through the skin puncture while the introducer remains inserted.

In embodiments, a sensor may be inserted via an actuator device including any of various electric, hydraulic, magnetic, pneumatic, or manual actuator devices including, for example, linear solenoid actuators, rotary solenoid actuators, CO₂ cartridge actuators, spring actuators, air pump actuators, etc.

FIG. 8A shows a cross-sectional view of a cantilevered introducer insertion device in accordance with various embodiments of the present invention. Prior to insertion, sensor 802 may be situated inside sensor insertion channel 804. Hollow introducer 806 may be molded into introducer insert molding 808 and the distal end of sensor 802 may be inside hollow introducer 806. Cantilever spring 810 may connect to the introducer via slot 818 in insert molding 808.

Referring now to FIG. 8B which shows the components of a cantilevered introducer insertion device without the device body, pin 812 may be attached to spring 810 via keyway 820. During manufacture, pin 812 may be inserted into keyway 820 and thereby locked into place. Spring 810 may be held inside slot 816 by an appropriate interference fit (see FIG. 8A).

Referring back to FIG. 8A, in an embodiment, triangular space 814 may provide room for spring 810 flexion. An actuator (not shown) may insert introducer 806 by applying force to pin 812 which may cause spring 810 to move inside triangular space 814 and force insert molding 808 forward. This action may insert introducer 806 into a patient's skin and cause a skin puncture. In an embodiment, sensor 802 may be subsequently forced through the interior of hollow introducer 806 and into the skin by entering through the skin puncture. In an alternative embodiment, sensor 802 may be inserted into the skin simultaneously with introducer 806 by entering through the inserted hollow introducer 806. After force is removed from pin 812, spring 810 may return to its resting position which may retract introducer 806, but not sensor 802, from the skin.

In an embodiment, a separate button may be mated to pin 812 for manual introducer insertion. In an embodiment, introducer insert molding 808 may be replaced by a cylindrical component with an appropriate slot for cantilevered spring attachment that may be attached to introducer 806 by means other than insert molding such as adhesive bonding or ultrasonic welding.

FIG. 9 shows a manual introducer pre-set actuator in accordance with an embodiment of the present invention. Sensor insertion button 902 and introducer insertion button 904 may be provided at the far end of actuator body 910 shown in a pre-insertion configuration. During insertion, a user may begin pushing on introducer insertion button 904 which will force introducer insertion rod 908 to move forward until its top surface is flush with the top surface of sensor insertion button 902. At that point, introducer insertion button 904 may encounter a stop (not shown) which may prevent further movement of introducer insertion rod 908. The patient may then begin pushing sensor insertion button 902 and force sensor insertion rod 906 forward until the end of its travel.

In embodiments, the end of introducer insertion rod 908 may contact the end of a pin (not shown) which may be in communication with an introducer (not shown). For the purposes of describing embodiments of the invention, the phrase “in communication with” means any of a variety of means of interaction between two or more elements whether direct or indirect. In this manner, pushing on introducer insertion button 904 may force the introducer to travel forward for the length of travel of introducer insertion button 904. In embodiments, this length of travel may be between 1 mm and 5 mm, for example 2 mm.

In embodiments, the end of sensor insertion rod 906 may contact the distal end of a sensor (not shown). In this manner pushing on sensor insertion button 902 may force the sensor to travel forward for the length of travel of sensor insertion button 902. In embodiments, this length of travel may be between 10 mm and 15 mm.

In embodiments, a single button may be utilized. The button may initially be in contact with an angled contact surface of an introducer insertion rod. Applying force to the button may initially cause the introducer insertion rod to force an introducer to puncture skin and be inserted a pre-determined distance therein. At that point, a release mechanism may allow the introducer insertion button to slip off of the angled contact surface which may halt the travel of the introducer. The button may then make contact with a sensor insertion rod which may force a sensor into the skin puncture created by the initial insertion of the introducer. The sensor may, in embodiments, be inserted into the skin a greater distance than the introducer.

In embodiments, a single button may be utilized along with a gearing mechanism to translate the motion of the button to both a sensor insertion rod and an introducer insertion rod. In embodiments, gear ratios for insertion of an introducer may be greater than the gear ratios for insertion of a sensor such as, for example, a 2:1 gear ratio for the introducer and a 1:1 gear ratio for the sensor. In embodiments, gear ratios other than 2:1 and 1:1 may be utilized. Due to the differing gear ratios, pressing the button may cause the introducer to travel a shorter distance than the sensor such as, for example, half as far. In embodiments, the initial positioning of the sensor and introducer may allow, upon application of force to the button, the introducer to pass through an opening of the device before the sensor passes through the opening. Thus, the sensor may be inserted into skin only after the introducer punctures the skin and is inserted a pre-determined distance therein.

In FIG. 10, manual introducer pre-set actuator 1010 is shown mated with cantilevered introducer insertion device 1000 in accordance with an embodiment of the present invention. As described in the discussion of FIG. 9 above, introducer insertion button 1004 may be pushed causing introducer insertion rod 1008 to move forward which may be in contact with pin 1012. Pin 1012 may then move forward, forcing spring 1014 and hollow introducer 1016 to move forward and introducer 1016 may then exit the bottom of device 1000 and puncture the skin of a patient onto whom the insertion device may be placed. Subsequently, sensor insertion button 1002 may be pressed causing sensor insertion rod 1006, which may be in contact with sensor 1018, to move forward. This may cause sensor 1018 to pass through sensor insertion guidance channel 1020, through hollow introducer 1016, and into the skin through the skin puncture.

Although a manual introducer pre-set actuator is shown mated to a cantilevered introducer insertion device in FIG. 10, it will be appreciated by one of ordinary skill in the art that in other embodiments alternative actuator devices may be mated to a cantilevered introducer insertion device. These alternatives include, for example, a rotating cam actuator or similar device. Additionally, any of various electric, hydraulic, magnetic, pneumatic, or manual actuator devices may be utilized including, for example, linear solenoid actuators, rotary solenoid actuators, CO₂ cartridge actuators, spring actuators, air pump actuators.

In an embodiment of the present invention, additional components may be housed in one or more separate modules that may be coupled to (for example, snapped to, wired to, or in wireless communication with) the various sensor insertion devices. For example, a separate module may contain a memory component, a battery component, a transmitter, a receiver, a transceiver, a processor, and/or a display component, etc.

In an embodiment of the present invention, a sensor with substantially uniform cross-section may be utilized. Alternatively, in an embodiment of the present invention, a sensor with a varied cross section may be used. In embodiments, a sensor may be cylindrical, squared, rectangular, etc. In an embodiment, a sensor may be a wire-type sensor. In an embodiment, a sensor may be flexible such that it may undergo normal flexion in an animal body without breaking, for hundreds or thousands of such flexions.

Likewise, in an embodiment of the present invention, an introducer with substantially uniform cross-section may be utilized. Alternatively, in an embodiment of the present invention, an introducer with a varied cross section may be used. In embodiments, an introducer may be cylindrical, squared, rectangular, etc.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. An insertion device comprising:
a housing having an opening and adapted to be placed in proximity to animal skin;
an analyte sensor guidance structure disposed inside the housing, said guidance structure adapted to allow passage of an analyte sensor through the guidance structure and through the opening of the housing upon application of a motive force to the analyte sensor; and
an introducer situated at least partially in the housing, said introducer adapted to be in communication with an actuator device, said introducer positioned to partially pass a predetermined distance through the opening of the housing upon application of motive force to the introducer by the actuator device, said introducer adapted to cause, upon application of the motive force, a skin puncture through which the analyte sensor can be inserted.

2. The insertion device of claim 1 further comprising an actuator device mated to the housing, said actuator device in communication with the introducer.

3. The insertion device of claim 1 wherein the introducer has at least a hollow portion and said introducer is adapted to allow an analyte sensor to pass through the hollow portion and exit the housing through the opening of the housing.

4. The insertion device of claim 1 wherein the introducer is a solid, non-hollow introducer.

5. The insertion device of claim 1 wherein said introducer is situated completely in the housing prior to the application of motive force to the introducer.

6. The insertion device of claim 1 wherein said introducer is partially extended from the housing prior to the application of motive force to the introducer.

7. The insertion device of claim 1 wherein said introducer is adapted to be inserted into skin prior to the insertion of the analyte sensor.

8. The insertion device of claim 1 wherein said introducer is adapted to be inserted into skin simultaneously with the insertion of the analyte sensor.

9. An insertion device comprising:
a housing having an opening and adapted to be placed in proximity to animal skin;
an analyte sensor guidance structure disposed inside the housing, said guidance structure adapted to allow passage of an analyte sensor through the guidance structure and through the opening of the housing upon application of a first motive force to the analyte sensor; and
an introducer situated at least partially in the housing, said introducer adapted to be in communication with an actuator device, said introducer positioned to partially pass a predetermined distance through the opening of the housing upon application of a second motive force to the introducer by the actuator device, said introducer adapted to cause, upon application of the motive force, a skin puncture through which the analyte sensor can be inserted.

10. The insertion device of claim 9 wherein said actuator device comprises a solenoid actuator.

11. The insertion device of claim 9 wherein said actuator device comprises a pressurized gas cartridge actuator.

12. The insertion device of claim 9 wherein said actuator device comprises a spring actuator.

13. The insertion device of claim 12, wherein said spring actuator comprises a cantilevered spring actuator.

14. The insertion device of claim 9 wherein both the first motive force and the second motive force are provided by the actuator device.

15. The insertion device of claim 9 further comprising another actuator device to provide the first motive force.

16. The insertion device of claim 9 wherein said introducer is situated completely in the housing prior to the application of motive force to the introducer.

17. The insertion device of claim 9 wherein said introducer is partially extended from the housing prior to the application of motive force to the introducer.

18. The insertion device of claim 9 wherein said introducer is adapted to be inserted into skin prior to the insertion of the analyte sensor.

19. The insertion device of claim 9 wherein said introducer is adapted to be inserted into skin simultaneously with the insertion of the analyte sensor.

20. A method for insertion of an analyte sensor comprising:
mounting, onto the skin of an animal, an insertion device, said insertion device comprising:
a housing having an opening and adapted to be placed in proximity to animal skin
an analyte sensor guidance structure disposed inside the housing, said guidance structure adapted to allow passage of an analyte sensor through the guidance structure and through the opening of the housing upon application of motive force to the analyte sensor; and
an introducer situated at least partially in the housing, said introducer adapted to be in communication with an actuator device, said introducer positioned to partially pass a predetermined distance through the opening of the housing upon application of motive force to the introducer by the actuator device, said introducer adapted to cause, upon application of the motive force, a skin puncture through which the analyte sensor can be inserted;
an analyte sensor positioned to pass through the guidance structure; and
applying a motive force to the introducer.

21. The method of claim 20 further comprising applying a motive force to the analyte sensor.

22. The method of claim 21 wherein the motive force applied to the introducer and the motive force applied to the analyte sensor are applied with the same actuator device.

23. The method of claim 21 wherein the motive force applied to the introducer and the motive force applied to the analyte sensor are applied with different actuator devices.
